SEQUENCE LISTING (5 sequences)

SEQ ID NO: 1 - DNA, 1384 bp, Artificial Sequence
  Description of Artificial Sequence pSK122 Plasmid
atcgattgctggtcgaaacgtccggctttctcaaaaggtttaccaatggcttccatcgcgcgagccgcatacggacgggaaagggttaactccggtttgcctttggcgaactctggagaggcggtgttatggcaatcggcacaacctaagttgttgacgatttccggaccgccgcgcgcccatttaccgtggaagtagccatcttcgccgtctttctggatcagacgcgccacatccgggcttttacaactccagcatgccatcggtagcggaccatcttcagcgtttttcggcgcaccggtacgcagggtttcacgcacatcggtcacagcaaaagcatgtccacgcggcttgttgtaatcgcgcgagaagggataccccgcccacaggatcaccagccgtggatcttccgccagggcgtcaacacgctctgactgttccgaggtggctttccaggagagatattgatcgggatgctgcggggcaaaggtttcattcttcgcttccacagttacaggttttgcgggagcagccgtttgttcagcgtgaacagaagtgaaaaagaaaaaaggaatcaataagctgaagatacggcgtgcgtttatttttatccttgtcataggggcttcatccgaattgcgccattgttgcaatggcggtttttattgtttttcacgacagtaaccgcacctacactgtcatgacattgctcgcccctatgtgtaacaaataaccacactgtgaatgttgtctttaatcaattgtaagtgcatgtaaaataccactttagagttagtcagtatcttcctctttttcaacagcatgcataactgcatgttcctcaaagaattaatcaacttttgttgctgaccttcaaaaattaccctgccgtttatttgcacaattctacttttgcgtgatctgtcgcccaaatactaaacaaaactgccaatacccctacatttaacgcttatgccacatattattaacatcctacaaggagaacaaaagcatgagccaaattcacaaacacaccattcctgccaacatcgcagaccgttgcctgataaaccctcagcagtacgaggcgatgtatcaacaatctattaacgtacctgataccttctggggcgaacagggaaaaattcttgactggatcaaaccttaccagaaggtgaaaaacacctcctttgcccccggtaatgtgtccattaaatggtacgaggacggcacgctgaatctggcggcaaactgccttgaccgccatctgcaagaaaacggcgatcgtaccgccatcatctgggaaggcgacgacgccagccagagcaaacatatcagctataaagagctgcaccgcgacgtctgccgcttcgccaataccctg

SEQ ID NO: 2 - DNA, 17 bp, Artificial Sequence
  Description of Artificial Sequence Primer
tcgaggtcgacggtatc

SEQ ID NO: 3 - DNA, 20 bp, Artificial Sequence
  Description of Artificial Sequence Primer
cgctctagaactagtggatc

SEQ ID NO: 4 - DNA, 20 bp, Artificial Sequence
  Description of Artificial Sequence Primer
gacgggaaagggttaactcc

SEQ ID NO: 5 - DNA, 23 bp, Artificial Sequence
  Description of Artificial Sequence Primer
tttggcccatggttttgttctcc

(19) United States
(12) Patent Application Publication
(10) Pub. No.: US 2004/0191861 A1
(43) Pub. Date: Sep. 30, 2004

(54) Method of protein synthesis

(75) Inventors: Jae-Gu Pan, Daejeon (KR); Heung-Chae Jung, Daejeon (KR); Sooan Shin, Daejeon (KR)

Correspondence Address: JONES DAY, 51 Louisiana Aveue, N.W, WASHINGTON, DC, 20001-2113, US

(73) Assignee: Korean Research Institute of Bioscience and Biotechnology

(21) Appl. No.: 10/608,078

(22) Filed: Jun. 30, 2003

Related U.S. Application Data
(63) Continuation of application No. 09/946,376, filed on Sep. 5, 2001, now abandoned.

(30) Foreign Application Priority Data
Sep. 5, 2000 (KR) 00-52464

(51) Int. Cl. C12P 21/02; C12N 1/21; C12N 15/74

(52) U.S. Cl. 435/69.1; 435/488; 435/252.33; 536/23.7

(57) ABSTRACT

The present invention relates to a method of protein synthesis and, more particularly, to a method of effective protein synthesis by regulating the expression of a protein in a host cell, wherein said host cell is transformed with an expression vector comprising a promoter as well as a DNA fragment for a gene that encodes a desired protein, wherein said promoter has an inductive activity for transcription during the resting stage of cell growth and also the induction of said protein expression can be controlled by varying culturing conditions.

### FIELD OF THE INVENTION

The present invention relates to a method of protein production and, more particularly, to a method of effective protein production by regulating the expression of a protein in a host cell, wherein the host cell is transformed with an expression vector comprising a promoter as well as a DNA fragment for a gene that encodes a desired protein, wherein the promoter is active at the resting stage of the culture and also the induction of said protein expression can be controlled by varying culturing conditions.

### BACKGROUND OF THE INVENTION

Protein synthesis via a microbial organism can be regulated mostly at the level of transcription and thus the selection of a most appropriate promoter that can strongly direct the synthesis of a desired protein is very important (Markrides, 1996, Microbial Reviews, 60, 512-538).

There are several factors that should be considered in selecting such a strong promoter. First, the promoter should have a very active transcriptional activity to synthesize sufficient amount of mRNA. Second, the promoter should be able to well control the protein expression, however, the promoter should not have any transcriptional activity or it should be kept at an extremely low level, if at all, prior to the induction of a given protein expression. Third, the promoter should be well transformed into a host cell. Finally, the promoter should have a relatively easy induction system for protein expression and is also preferred to be cost-effective.

There are a number of promoters that have been used in constructing recombinant expression vectors for protein biosynthesis using E. coli as a host cell; e.g., lac promoter (Roberts et al., 1979, Proc. Natl. Acad. Sci. USA, 76, 760-764), tac promoter (Aman et al., 1983, Gene, 25, 167-178), trc promoter (Brosius et al., 1985, J. Biol. Chem., 260, 3539-3541), PL or PR promoter (Elvin et al., 1990, Gene, 87, 123-126), and T7 promoter (Studier et al., 1986, J. Mol. Biol., 189, 113-130). These promoters are well known to exhibit very strong transcriptional activities in the presence of a particular inducer and accumulate more than 10-30% of the total proteins in cells. However, these promoters are considered disadvantageous in that their transcriptional activities are maintained at a relatively high level when cells are at a normal growth stage. Further, recombinant expression systems utilizing lac promoter or promoters derived from lambda phage are very effective and convenient in culturing E. coli for a general laboratory scale use, however, they are not well suited for production in the large culture. Still further, expression systems with lac promoter use isopropyl-β-D-thiogalactoside (IPTG) as an inducer, a highly expensive compound, and thus it becomes quite costly to prepare a large-scale cell culture. In case of an expression system using a promoter derived from lambda phage, it is required to increase a temperature for the expression of a protein and this increase in temperature results in generation of inactive inclusion bodies. Also, uniform temperature condition can be hardly maintained within a culture when preparing a large-scale culture.

Various efforts have been reported to solve the above-mentioned problems; e.g., phoA promoter (Miyake et al., 1985, J. Biochem., 97, 1429-436), cst-1 promoter (Turner et al., 1992, Biotechnol. Bioengin., 40, 271-79), nar promoter (Lee et al., 1996, Biotechnol. Lett., 18, 129-134), and trp promoter (Yansura et al., 1990, Methods. Enzymol., 185, 54-0). However, these promoters are not advantageous in that the regulation of expression is very complicated and inefficient.

### SUMMARY OF THE INVENTION

To solve the above problems, the inventors of the present invention focused their studies on developing a protein expression system with great efficiency and convenience and the biosynthesis of a protein using this system thereof. As a result, the inventors invented a novel protein expression method creating a expression vector having a promoter which has a transcriptional activity during the resting stage of the cell culture so that protein expression can be induced only at the resting stage of the culture, and also the transcription can be induced in the presence of an organic acid compound in the culture medium such as acetic acid or succinic acid.

Therefore, the object of the present invention is to provide a new method of protein synthesis by means of a protein expression system which is characterized in that the system contains a promoter which has a transcriptional activity during the resting stage of cell culture and also a protein expression is regulated with ease as well as efficiency.

Another object of the present invention is to provide a recombinant vector comprising a gene that encodes a desired protein and a promoter which has an inductive activity for transcription during the resting stage of cell growth, wherein the transcription is induced by an organic acid compound.

Yet, another object of the present invention is to provide a recombinant transformant containing the said vector directing a desired protein synthesis.

### BRIEF DESCRIPTION OF THE DRAWINGS

FIG. 1 is a schematic diagram showing the process of constructing pSS112, an expression vector, according to the Example 1 of the present invention wherein a DNA fragment that includes an acs gene and its promoter obtained from Kohara lambda library 638 of E. coli chromosome subcloned into pGEM-7fz(+/−).

FIG. 2 shows the results of SDS-PAGE performed according to the Example 2 of the present invention showing the level of protein ACS (indicated by arrows) synthesized in JM109 host cells transformed with pSS112.

FIG. 3 is a schematic diagram showing the process of constructing pSK122(4.4 kb) according to the Example 3 of the present invention, wherein a 1384 bp DNA fragment with an acs promoter region as well as a region that encodes the beginning part of acs gene is cleaved out from pSS112 and subcloned into pBluescript IT KS(+/−).

FIG. 4 is a schematic diagram showing the process of constructing pSS121(about 11.4 kb) according to the Example 3 of the present invention, wherein a XhoI-EcoRV DNA fragment of the pSK122 in FIG. 3 is cleaved out, filled in by Klenow filling reaction and is then subcloned into pRS415.

FIG. 5 shows the results of SDS-PAGE performed according to the Example 4 of the present invention depicting the level of β-glucosidase (indicated by arrows) synthesized by the induction of acs promoter in JM109 host cells transformed with pSS121 by culturing in LB medium.

FIG. 6 shows the result of SDS-PAGE performed according to the Example 4 of the present invention depicting the level of β-glucosidase (indicated by arrows) synthesized by the induction of acs promoter in JM109 host cells transformed with pSS121 by culturing in LB medium wherein the LB medium is added with glucose.

FIG. 7 shows the results of SDS-PAGE performed according to the Example 4 of the present invention depicting the level of β-glucosidase (indicated by arrows) synthesized by the induction of acs promoter in JM109 host cells transformed with pSS121 by culturing in M9 glucose medium (samples 1-4) and M9 succinic medium (samples 5-8).

FIG. 8 is a schematic diagram showing the process of constructing pJHC30, a general expression vector constructed by subcloning only acs promoter, as well as pJHC31 and pJHC35 according to the Example 5 of the present invention, wherein chitinase gene and lipase gene are subcloned, respectively.

FIG. 9 is the result of SDS-PAGE performed according to the Example 5 of the present invention showing the level of chitinase (indicated by an arrow) expressed by pJHC31.

FIG. 10 is the result of SDS-PAGE performed according to the Example 5 of the present invention showing the level of lipase (indicated by an arrow) expressed by pJHC35.

### DETAILED DESCRIPTION OF THE PREFERRED EMBODIMENTS

The present invention relates to a method of producing a desired protein by gene recombination, the method comprising the steps of:

(a) constructing a gene expression vector comprising a gene that encodes the desired protein and a promoter which has an inductive activity for transcription during the resting stage of cell growth, wherein the transcription is induced by an organic acid compound;

(b) introducing said gene expression vector into a host cell;

(c) inducing the expression of said desired protein by culturing said host cell in culture medium; and

(d) recovering the desired protein.

The present invention is described in detail as set forth hereunder.

The inventors of the present invention, considering the importance of selection of a suitable promoter in protein expression technology, studied acs promoter of E. coli based on the report by Kumari et al. (Kumari et al, 1995, J. Bacteriol., 177, 2878-2886) and subsequently revealed that the expression of acs promoter is inhibited by the glucose present in the culture medium but induced by acetic acid, and this kind of induction of expression is regulated during the stationary phase of cell culture (Shin et al, 1997, FEMS Microbiology Letters, 146, 103-08; Kumari et al., 2000, J. Bacteriol., 182(2):551-554).

The inventors of the present invention introduced acs promoter into an expression system in order to utilize the advantage of a promoter that has a transcriptional activity during the resting stage of a host cell growth. Using this kind of a promoter enables to distinguish the steps of cell growth from the steps of protein expression. By differentiating these two different categories of steps, an optimized culturing strategy most suitable for each step can be established and also protein expression can be induced by culturing host cells at a high concentration thus resulting in a more efficient protein synthesis.

For the expression of a protein during the resting stage of a host cell growth, it is required to provide an appropriate level of energy as well as a method toward a long-term protein synthesis, and the recent high protein expression system during the resting stage of a host cell growth suggested a good resolution to overcome the long-awaited problem (Rowe & Summers, 1999, Appl. Environ. Microbiol., 65, 2710-2715).

The method of constructing a recombinant expression vector according to the present invention is described in detail as described below.

First, it is required to obtain a promoter wherein the transcription of the promoter can be induced by an organic acid compound such as acetic acid or succinic acid. The promoter of the present invention is characterized in that it includes a DNA fragment of acs gene of E. coli or a DNA fragment that is partially the same as that of acs gene of E. coli in nucleotide sequence or a DNA fragment wherein its biological function is similar to that of acs gene of E. coli. This transcriptional regulatory region of the acs gene of E. coli is present upstream region of the gene, up to 391 bp from translational start codon of it, which is located between nrfA and acs genes (Kumari et al., 2000, J. Bacteriol., 182(2):551-554). Further, acs promoters derived from other bacteria in addition to E. coli, fungi, yeasts or actinomyces can be also easily applied if they can serve the same function.

Second, it is required to obtain a DNA fragment that encodes a useful protein by using a conventional method. The DNA fragments used in the present invention include those which contain acs gene that encodes acetyl Co A synthetase, lac Z gene that encodes β-galactosidase, chiA gene that encodes chitinase, tliA gene that encodes lipase and other DNA fragments which contain genes for proteins that need to be expressed for conventional purposes can be also utilized. The desired proteins expressed by the present invention could be one selected from the group consisting of hormones, hormone analogs, enzymes, enzyme inhibitors, receptors or their fragments, antibodies or their fragments, single-chain antibodies, structural proteins, toxin proteins, and plant defense-inducing molecules.

Third, it is required to construct a recombinant expression vector by ligating the above promoter and a foreign protein by a conventional recombinant DNA technology so that the selected foreign protein can be exclusively or almost exclusively expressed by the promoter.

Fourth, it is required to transform thus constructed expression vector into a host cell for stability purpose. The host cells that can be used in the present invention are bacteria that belong to Gram-negative bacteria. Particularly, the host cells could be Enterobacteriaceae such as E. coli. The transformed host cells are cultured by using a conventional method.

The present invention provides a method to effectively synthesize a protein by regulating the expression of the above expression system depending on the varying culturing conditions. That is, the present invention is well characterized in that it can easily regulate the expression of a foreign protein by means of a culture medium unlike the conventional methods which use either IPTG as an inducer or use temperature increase. The culture media to be used are selected from the group consisting of a complex medium, a minimal culture medium containing acetic acid or succinic acid as a sole carbon source, or a minimal medium containing glucose or glycerol as a sole carbon source.

The preferred examples of methods to induce the expression of foreign proteins using the above-mentioned culture media include:

(a) a method to induce a constituitive expression by culturing a transformed host cell in a complex medium containing yeast extract, peptone, amino acids, vitamins, etc., without using an inducer;

(b) a method to induce a constituitive expression during the cell growth stage or the resting stage of a host cell growth by culturing a transformed host cell in a minimal medium containing acetic acid or succinic acid as a sole carbon source without using an inducer;

(c) a method to induce an expression by culturing a transformed host cell in a minimal medium containing glucose or glycerol as a sole carbon source by adding an organic acid compound acetic acid or succinic acid as an inducer during a desired stage of cell growth; and

(d) a method to induce a spontaneous expression during the resting stage of cell growth by culturing a transformed host cell in a minimal medium containing a sugar such as glucose or glycerol as a sole carbon source without using an inducer.

As described above, an organic acid compound such as acetic acid or succinic acid can serve as an inducer for protein expression from the early stage of culture by using these as a carbon source (b), or alternatively by initially culturing in a medium wherein no or extremely low amount of an organic acid compound is contained and then adding 0.01%˜0.5% (w/v) of the above organic acid at a later desired stage thus inducing the expression of a foreign protein (c). The examples of an inducer that can be used for the expression, in addition to acetic acid or succinic acid, are organic acid compounds such as maleic acid, fumaric acid, and citric acid.

When using glucose as a carbon source, the expression of a foreign protein is inhibited during the cell growth stage, and acetic acid, a byproduct from a cell growth stage, serves as an inducer for expression by maintaining a cell culture for over 24 hrs (d). The examples of a sugar that can be used in the medium for the expression, in addition to glucose, are sugars for fermentation carbon and energy sources such as glycerol, fructose and maltose.

The above complex media include LB medium and other conventional media such as YT medium (tryptone, 8 g/L; yeast extract, 5 g/L; NaCl, 5 g/L), 2X YT medium, B broth or tryptone broth (tryptone, 10 g/L; NaCl, 8 g/L), Luria broth (tryptone, 10 g/L; yeast extract, 5 g/L; NaCl, 0.5 g/L), and the above minimal media are M9 minimal medium, M63 minimal medium (KH2PO4, 13.6 g/L; (NH4)2SO4, 2 g/L; FeSO4×7H2O, 0.5 mg/L; adjust to pH 7.0 using KOH).

The advantages of the protein expression according to the present invention can be summarized as follows.

First, the inducers of the present invention are much cheaper than IPTG and thus the present invention is cost-effective. Second, there are various kinds of inducers; inducers are not much affected by the impurities in performing the desired induction; and also instant induction of expression in a large-scale cell culture is also possible. Third, the expression can be carried out in the absence of a particular inducer during the resting stage of cell growth; in particular, the expression during the resting stage of cell growth is advantageous in that more soluble proteins can be expressed by inhibiting the generation of inclusion bodies resulted from overexpression of a given protein. Besides, a precise control of inhibition or regulation of expression is also possible.

In the present invention, the protein expression is proceeded using a conventional method while still preserving their own activities of expressed proteins, and the process is completed by passing through separation and purification.

Hereunder is given a detailed description of the present invention using the following examples, however, it is appreciated by those skilled in the art that the present disclosure of the preferred form has been made only by way of examples and that numerous changes in the details of the construction, combination, and arrangement of parts may be resorted to without departing from the spirit and scope of the invention. In particular, those proteins as acetyl Co-A (ACS), β-galactosidase (Lac Z), chitinase (Chi A) and lipase (Tli A) used as target proteins are only several examples for the completion of the present invention and they should not be construed as limiting the scope of the present invention.

### EXAMPLE

The materials and methods used in the examples of the present invention are as set forth hereunder.

First, E. coli JM109 was used as a host cell and pGEM-7Zf(+/−) (Promega, USA), pBluescript II-KS(+/−) (Stratagene, USA), pTrc99A (Pharmacia, Sweden) and pRS41 (Simons et al., 1987, Gene, 53, 85-96) were used in subcloning acs promoter.

Second, LB medium (yeast extract 5 g/L, bactotryptone 10 g/L, NaCl 5 g/L, pH 7.2) was used as a basic complex medium, and acetic acid, succinic acid and glucose were added at the concentration of 0.2(w/v) to the M9 minimal medium (Na₂HPO₄ 6 g/L, KH₃PO₄ 3 g/L, NaCl 0.5 g/L, NH₄Cl 1 g/L, and add 10 mL of 0.01M CaCl₂ after vapor sterilization, pH 7.2) when using one of those as a carbon source. When necessary, antibiotics of ampicillin and tetracycline were used at the concentration of 100 μg/mL and 15 μg/mL, respectively, and cells were cultured at 37° C.

LB culture medium was prepared by using GFBCO BRL LB broth base (Cat. 12780-052, Life Technologies Co., Ltd., USA) and M9 medium was prepared by using M9 minimal salt (Cat. M6030, Sigma Co, Ltd., USA).

Enzymes including restriction enzymes used in DNA cloning were purchased from Korea Postech Co., Ltd. and T4 ligase used was purchased from Boeringer-Mannheim Co., Ltd. (Germany). Desired DNA fragments were recovered from agarose gels by using QiaEx II Gel Extraction Kit (Qiagen Co., Ltd., Germany).

The methods used in the present invention for the manipulation of recombinant DNA and the analysis of total proteins produced by cells were performed according to the methods described in ‘Molecular Cloning’ by Sambrook et al (A Laboratory Manual, 3rd ed., 2001, Cold Spring Harbor Laboratory Press, USA), unless otherwise specified.

Soluble and insoluble cellular proteins were isolated as follows. First, 1 mL of E. coli cell culture was centrifuged at 12000 rpm for 5 min, and the resulting pellet was washed with 0.85% (w/v) a saline solution and resuspended in a cell-homogenizing buffer solution [50 mM Tris-Cl (pH 8.0), 1 mM PMSF (phenylmethanesulfonyl fluoride)]. The resuspended cells were then homogenized using an ultrasonic cell-homogenizer and centrifuged at 12,000 rpm for 10 min. The resulting supernatant was analyzed as a source for a soluble protein while the lower fraction was analyzed as a source for an insoluble protein.

### Example 1

### Construction of pSS112 Having an E. coli DNA Fragment for Acetyl Co-A Synthetase Gene (acs) and acs Promoter

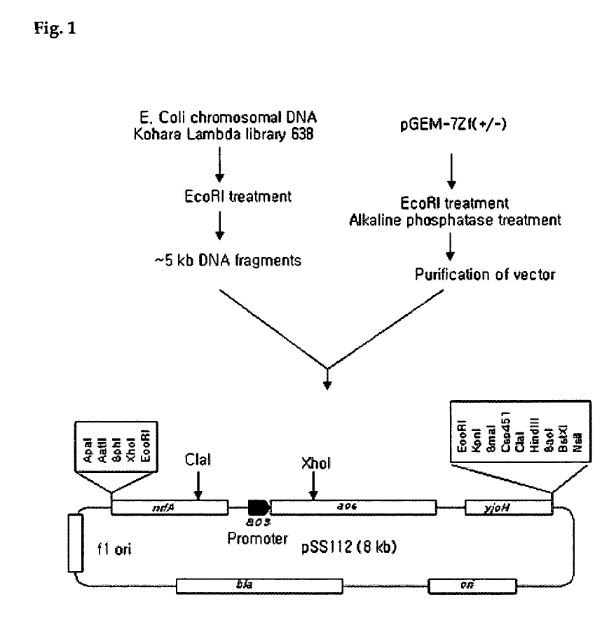

Kohara lambda library no. 638 (Kohara et al., 1989, Cell, 50, 495-508), a known E. coli chromosome library, was used as a DNA fragment for acs gene and acs promoter. The DNA fragment was digested with EcoRI and a resulting DNA fragment about 5 kb in size was separated out and subcloned into pGEM-7Zf(+/−) (Promega, USA), which was also digested with EcoRI, to construct pSS112 (FIG. 1). JM109, an E. coli cell line, was transformed by using thus constructed pSS112, and the transformed JM109/pSS112 was cordially deposited with the Korea Research Institute of Bioscience and Biotechnology Korean Collection for type cultures, Korean Deposit Associates, located in #52, Oun-dong, Yusung-gu, Daejon, 305-333, Republic of Korea, on Oct. 21, 1999, under Deposit Accession Number KCTC 067BP.

### Example 2

### Method of Acetyl Co-A Synthetase (ACS) via acs Promoter

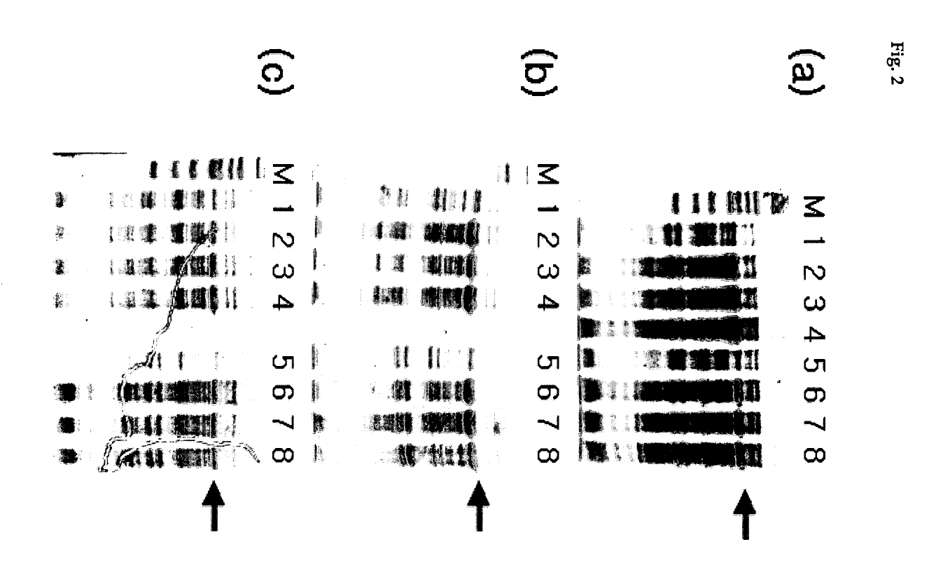

Since pSS112 contains a gene that encodes acetyl Co-A synthetase and a promoter that is involved in the expression of the acetyl Co-A synthetase, this Example focused on the confirmation of the expression of acetyl Co-A synthetase by culturing the transformant JM109/pSS112 in LB medium, a complex medium, or in semi-synthetic M9 minimal culture medium containing 0.2% (w/v) casaminoacid and 0.2% (w/v) glucose at 37° C. (FIG. 2). The result of protein expression was analyzed in SDS-PAGE; more specifically, FIG. 2(a) shows the total cumulative proteins synthesized by the transformant JM109/pSS112, FIG. 2(b) shows the result for a soluble protein fraction and FIG. 2(c) shows the result for an insoluble protein fraction.

Here, M is a protein size marker, 1 represents the analysis for a sample obtained from a 4 hr culture in LB medium, 2 for a sample obtained from a 7 hr culture in LB medium, 3 for a sample obtained from an 11.2 hr culture in LB medium, 4 for a sample obtained from a 22 hr culture in LB medium, 5 for a sample obtained from a 4 hr culture in M9 succinic medium, 6 for a sample obtained from a 7 hr culture in M9 succinic medium, 7 for a sample obtained from an 11.2 hr culture in M9 succinic medium, and 8 for a sample obtained from a 22 hr culture in M9 succinic medium.

The protein ACS, expressed by its own promoter without any chemical inducer or a temperature increase, was expressed at an extremely low level during the first 4 hr of the log phase of cell growth when cultured in LB medium, however, the expression was drastically raised during resting stage by reaching almost 40% of the total protein. When cultured in semi-synthetic M9 minimal medium having glucose as a carbon source, the expression of ACS protein was kept at a low level during the log phase of cell growth and about 28% of the total protein was accumulated within a cell during the resting stage. Therefore, it is shown that all the proteins expressed by acs promoter undergo a large-scale expression during the resting stage of cell growth.

### Example 3

### Construction of pSS121

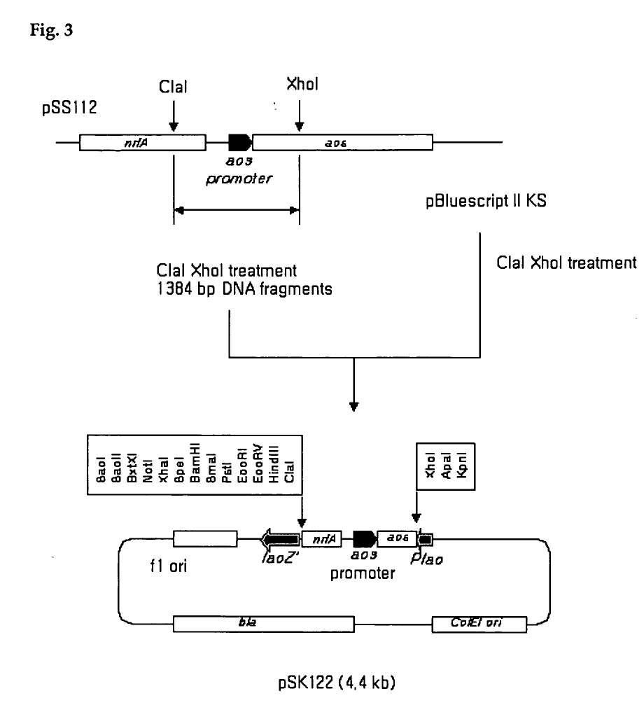

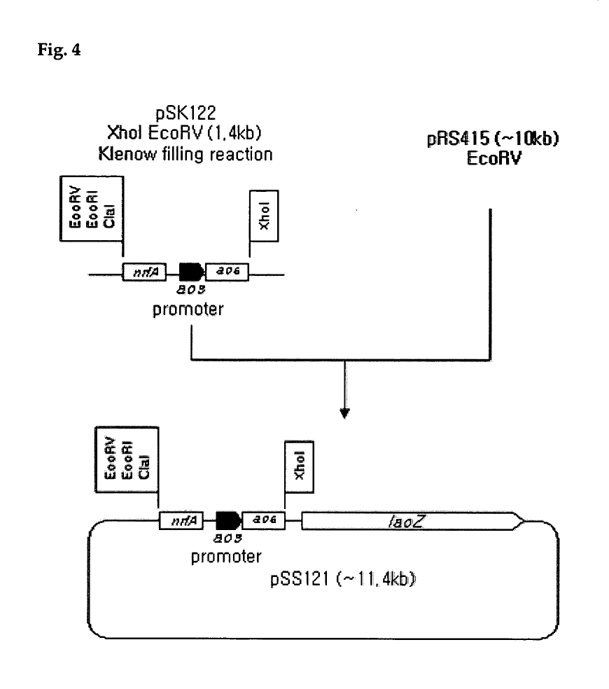

For the expression of β-galactosidase, a protein often used for test of expression, pSS121 was constructed as described below (FIGS. 3 and 4). A DNA fragment containing acs promoter, a 1384 bp fragment of pSS112 digested with Cla I and XhoI, was subcloned into pBluescript II (KS), which was also digested with the same restriction enzymes, and pSK122 was subsequently constructed. Here, the promoter region of pSK122 (SEQ ID NO:1) was sequenced to analyze the sequences of both the 5′strand and the 3′strand of pSK122 by using KS primer (SEQ ID NO:2) and SK primer (SEQ ID NO:3), respectively (Stratagene Co., Ltd., USA). Sequence analysis reaction was performed by using Big Dye Terminater Sequencing Kit (Perkin Elmer Co., Ltd., USA) and the result was analyzed by using a sequence analyzer (Model 377, Stratagene Co., Ltd., USA) and this sequence reading was repeated three times for more accurate identification of given sequences. Then, pSK122 was digested with XhoI and EcoRV, and the digested DNA fragments became blunt-ended by Klenow filling reaction. A 1.34 kb DNA fragment was isolated, purified and subcloned into pRS415, which was digested with EcoRV. A plasmid comprising acs promoter and β-galactosidase gene in this order was selected by a restriction map and was named pSS121 accordingly. Therefore, pSS121 became a plasmid vector having a genetic structure wherein the expression of lac Z gene can be induced by acs promoter. Finally, JM 109, an E. coli cell line, was transformed by using thus constructed pSS121, and the transformed JM109/pSS121 was cordially deposited with the Korea Research Institute of Bioscience and Biotechnology Korean Collection for type cultures, Korean Deposit Associates, located in #52, Oun-dong, Yusung-gu, Daejon, 305-333, Republic of Korea, on Oct. 21, 1999, under Deposit Accession Number KCTC 0675BP.

### Example 4

### High Expression of β-Galactosidase by acs Promoter

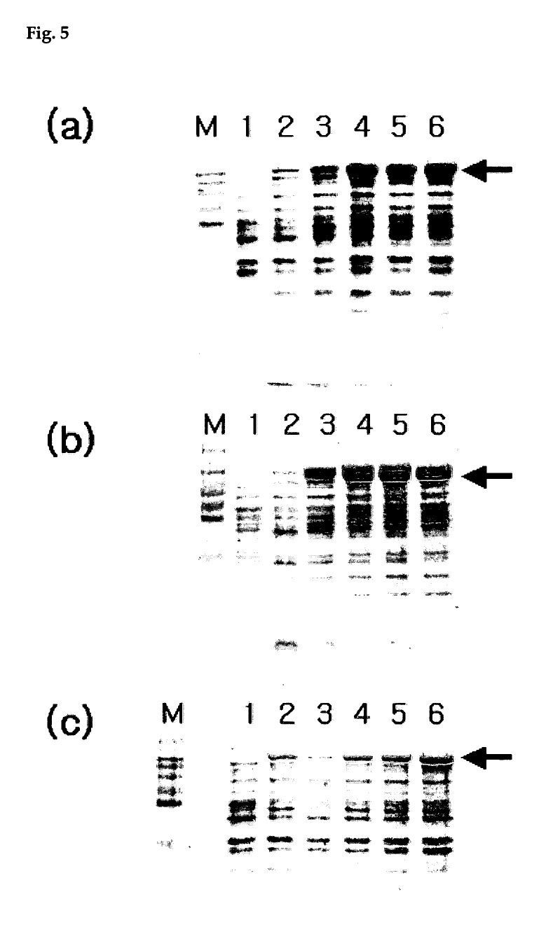

JM109/pSS121, an E. coli cell line transformed with pSS121, was cultured in LB medium, a complex medium, and the protein expression was performed as in Example 2 during the resting stage of cell growth. As a result, the level of β-galactosidase synthesis induced by acs promoter was analyzed on SDS-PAGE (FIG. 5). FIG. 5(a) shows the total cumulative proteins synthesized by the recombinant JM109/pSS112, FIG. 5(b) shows the result for a soluble protein fraction and FIG. 5(c) shows the result for an insoluble protein fraction.

Here, M is a protein size marker, 1 represents a sample of the total protein of JM109, 2 for a sample obtained from a 4 hr culture of JM109/pSS112 in LB medium, 3 for a sample obtained from a 6.5 hr culture in LB medium, 4 for a sample obtained from a 9 hr culture in LB medium, 5 for a sample obtained from an 11 hr culture in LB medium, and 6 for a sample obtained from a 24 culture in LB medium.

As shown in FIG. 5, more amount of the enzyme, β-galactosidase, was expressed during the resting stage (after 9 hr), than during the first 4 hr of log phase and the amount of protein expression accounted for approximately 50% of the total cellular protein. When the expressed proteins were fractionized into a soluble protein and an insoluble protein, it was shown that most proteins were expressed in cells in a soluble form.

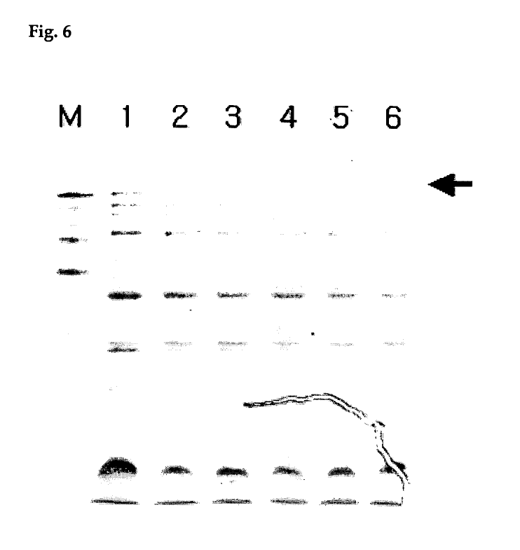

To investigate whether the expression of β-galactosidase can be regulated when the β-galactosidase expression is induced by the acs promoter, the protein expression was analyzed by adding 4 g/L of glucose into an LB medium (FIG. 6). The level of β-galactosidase synthesis induced by the acs promoter is analyzed on SDS-PAGE as shown in FIG. 6.

Here, M is a protein size marker, 1 represents a sample obtained from a 2 hr culture, 2 for a sample obtained from a 4.5 hr culture, 3 for a sample obtained from a 5.5 hr culture, 4 for a sample obtained from a 7.5 hr culture, 5 for a sample obtained from an 11 hr culture, and 6 for a sample obtained from a 24 culture.

As shown in the result, the expression cultured in complex LB medium was completely inhibited when glucose was added.

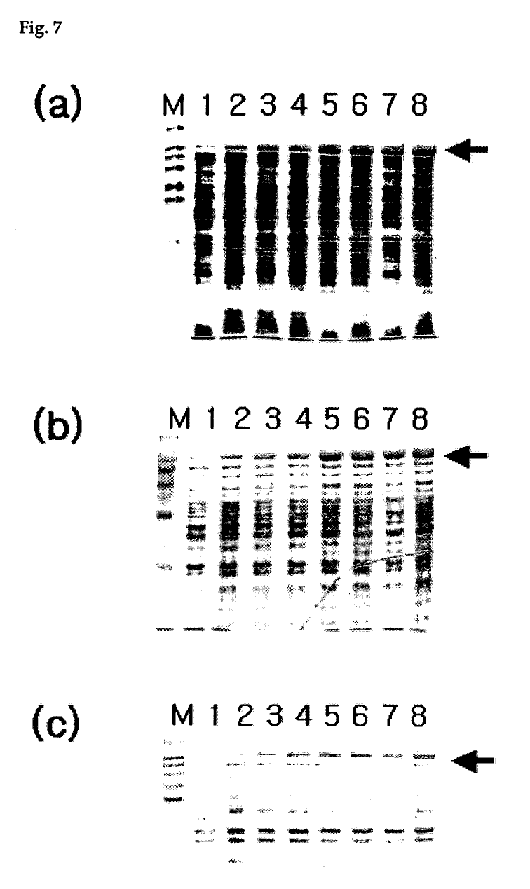

FIG. 7 shows the amount of proteins synthesized when cultured in M9 minimal culture medium wherein glucose or succinic acid was used as a sole carbon source. E. coli JM109 transformed with pSS121 was cultured in two different medium conditions of an M9 glucose medium (samples 1-4) and in an M9 succinic medium (samples 5-8), and the level of β-galactosidase synthesis induced by acs promoter as indicated by an arrow was analyzed on SDS-PAGE.

Here, FIG. 7(a) shows the total cumulative proteins synthesized by the recombinant JM109/pSS112, FIG. 7(b) shows the result for a soluble protein fraction and FIG. 7(c)? shows the result for an insoluble protein fraction. Also, M is a protein size marker, 1 represents a sample obtained from a 4.5 hr culture in M9 glucose medium, 2 for a sample obtained from a 7 hr culture in M9 glucose medium, 3 for a sample obtained from a 12 hr culture in M9 glucose medium, 4 for a sample obtained from a 20 hr culture in M9 glucose medium, 5 for a sample obtained from a 4.5 hr culture in M9 succinic medium, and 6 for a sample obtained from a 7 hr culture in M9 glucose medium, 7 for a sample obtained from a 12 hr culture in M9 succinic medium, 8 for a sample obtained from a 20 hr culture in M9 succinic medium. As shown in FIG. 7, it was also revealed that protein expression was remarkably inhibited when using glucose as a sole carbon source.

The amount of protein synthesis was observed to be less than 2% of the total protein during the log phase, and approximately 10% of the total protein was accumulated in a cell during the resting stage. When succinic acid was used as a carbon source, in contrast, more than 40% of the total protein was accumulated during the initial culturing stage of log phase and resting stage, and the proteins expressed were mostly soluble proteins.

### Example 5

### Construction of an Expression Vector pJHC30 for a Foreign Protein and the Expression of the Foreign Protein Induced by acs Promoter

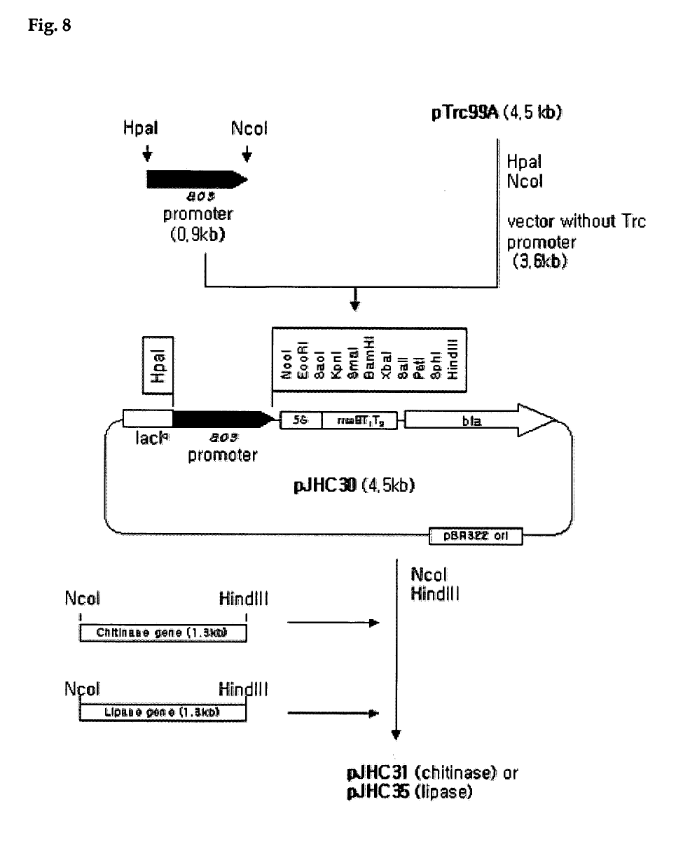

For the application of the expression system of the present invention to an expression of a foreign protein, an expression vector pJHC30 was constructed by replacing trc promoter present in pTrc99A, a conventional highly expressive vector for E. coli, with acs promoter by subcloning acs promoter via PCR technology and its usefulness was examined. In subcloning acs promoter, pSS112, which was used as a DNA template for acs promoter, was PCR amplified by using SEQ ID NO:4 and SEQ ID NO:5 were used as primers, respectively, digested with Hpa I and NcoI and then subcloned into pTrc99A, also digested with the same restriction enzymes (FIG. 8). Thus constructed pJHC30 was transformed into JM109 and the resulting JM109/pSS121 was cordially deposited with the Korea Research Institute of Bioscience and Biotechnology Korean Collection for type cultures, Korean Deposit Associates, located in #52, Oun-dong, Yusung-gu, Daejon, 305-333, Republic of Korea, on Dec. 15, 1999, under Deposit Accession Number KCTC 0712BP. However, the use of pTrc99A is intended to exemplify the usefulness of acs promoter and the use of a useful vector thus should not be restricted to pTrc99A.

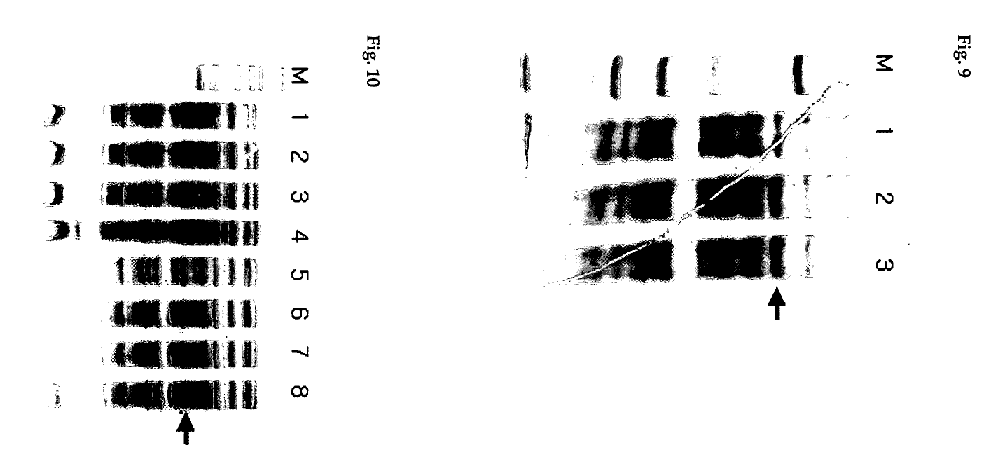

As a step toward the expression of a protein by acs promoter, about 1.3 kb chitinase gene derived from Serratia marcescens ATCC 27117 and about 1.3 kb of lipase gene derived from Peudomonas fluorescens SIK W1 (Ahn et al, 1998, J. Bacteriol., 181, 1847-1852) were subcloned into pJHC31 and pJHC35 (FIG. 8), respectively. The above pJHC31 and pJHC35 were transformed into E. coli JM109 and proteins were expressed. The results were analyzed on SDS-PAGE as shown in FIGS. 9 and 10.

In FIG. 9, M is a protein size marker, 1 represents a sample of total JM109 protein, 2 for a sample obtained from a 20 hr culture in LB medium, 3 for a sample obtained from a 20 hr culture in M9 minimal succinic medium, 4 for a sample obtained from a 7.5 hr culture in LB medium, 5 for a sample obtained from an 11 hr culture in LB medium, and 6 for a sample obtained from a 24 hr culture in LB medium.

FIG. 10 shows the result of lipase expression via pJHC35 analyzed on SDS-PAGE. In FIG. 10, M is a protein size marker, 1 represents a sample obtained from a 7 hr culture in LB medium, 2 for a sample obtained from a 9 hr culture in LB medium, 3 for a sample obtained from a 12 hr culture in LB medium, 4 for a sample obtained from a 24 hr culture in LB medium, 5 for a sample obtained from a 6.5 hr culture in M9 succinic medium, 6 for a sample obtained from an 8.5 hr in M9 succinic medium, 7 for a sample obtained from a 12.5 hr culture in M9 succinic medium, 8 for a sample obtained from a 24 hr culture in M9 succinic medium.

As described earlier, the protein expression reached about 16% of the total protein of E. coli in case of chitinase while the protein expression reached about 5% in the case of lipase, which is generally not well expressed in E. coli.

As described above, the protein expression system of the present invention enables a transformed host cell to effectively synthesize a protein. Further, the method to induce protein expression of the present invention is not only able to regulate the expression with an added precision but is also shown advantageous in that the inducers are various in its kinds, the inductive activities are not affected by the impurities contained, and they are also able to induce an instant expression even in a large-scale cell culture. More specifically, the method of the present invention can induce protein expression during the resting stage of cell growth thus preventing the generation of inclusion bodies and expressing more amount of a soluble protein.

What is claimed is:

1. A method of producing a desired protein by gene recombination, the method comprising the steps of:
(a) constructing a gene expression vector comprising a gene that encodes the desired protein and a promoter which has an inductive activity for transcription during the resting stage of cell growth, wherein the transcription is induced by an organic acid compound;
(b) introducing said gene expression vector into a host cell;
(c) inducing the expression of said desired protein by culturing said host cell in culture medium; and
(d) recovering the desired protein.

2. The method according to claim 1, wherein said organic acid compound is one selected from the group consisting of acetic acid, succinic acid, maleic acid, fumaric acid and citric acid.

3. The method according to claim 1, wherein said promoter includes 1 kb upstream DNA fragment of acs gene of E. coli.

4. The method according to claim 3, wherein said acs promoter is derived from bacteria, fungi, yeasts or actinomyces.

5. The method according to claim 1, wherein said gene contains a DNA fragment that encodes any one selected from the group consisting of hormones, hormone analogs, enzymes, enzyme inhibitors, receptors or their fragments, antibodies or their fragments, single-chain antibodies, structural proteins, toxin proteins, and plant defense-inducing molecules.

6. The method according to claim 5, wherein said gene contains a DNA fragment selected from the group consisting of acs gene that encodes acetyl Co A synthetase, lac Z gene that encodes β-galactosidase, chiA gene that encodes chitinase or tliA gene that encodes lipase.

7. The method according to claim 1, wherein said host cell is Gram-negative bacteria.

8. The method according to claims 1, wherein said culture medium is one selected from the group consisting of a complex medium, a minimal culture medium containing acetic acid or succinic acid as a sole carbon source, and a minimal medium containing glucose or glycerol as a sole carbon source.

9. The method according to claim 8, wherein said culture medium is the minimal medium containing glucose or glycerol as a sole carbon source and wherein said culture medium contains either acetic acid or succinic acid as an inducer.

10. A vector comprising a gene that encodes a desired protein and a promoter which has an inductive activity for transcription during the resting stage of cell growth, wherein the transcription is induced by an organic acid compound.

11. The vector according to claim 10, wherein said organic acid compound is one selected from the group consisting of acetic acid, succinic acid, maleic acid, fumaric acid and citric acid.

12. The vector according to claims 10 or 11, wherein said promoter includes 1 kb upstream DNA fragment of acs gene of E. coli.

13. The vector according to claim 12, wherein said acs promoter is derived from bacteria, fungi, yeasts or actinomyces.

14. The vector according to claim 10, wherein said gene contains a DNA fragment that encodes one selected from the group consisting of hormones, hormone analogs, enzymes, enzyme inhibitors, receptors or their fragments, antibodies or their fragments, single-chain antibodies, structural proteins, toxin proteins, and plant defense-inducing molecules.

15. A transformed cell comprising the vector according to any one of claims 10 to 14.

16. The cell according to claim 15, wherein the cell is Gram-negative bacteria.
